(19) United States
(12) Patent Application Publication
(10) Pub. No.: US 2020/0085551 A9
(43) Pub. Date: Mar. 19, 2020

(54) DENTAL SUCTION ARRANGEMENT

(71) Applicant: Thomas Asum, Göteborg (SE)

(72) Inventor: Thomas Asum, Göteborg (SE)

(21) Appl. No.: 15/848,919

(22) Filed: Dec. 20, 2017

Related U.S. Application Data
(63) Continuation-in-part of application No. PCT/EP2016/064618, filed on Jun. 23, 2016.
Application No. PCT/EP2016/064618 is a continuation-in-part of application No. 15/538,943, filed on Jun. 22, 2017.

(30) Foreign Application Priority Data
Jun. 26, 2015 (SE) 15508930
Dec. 20, 2016 (EP) 16205406.8

(51) Int. Cl. A61C 17/06; A61F 13/36; A61M 1/00; A61M 25/00; A61C 5/90; A61C 13/15

(52) U.S. Cl. CPC A61C 17/043; A61F 13/36; A61M 1/008; A61F 2013/4506; A61C 5/90; A61C 19/003; A61M 25/007

(57) ABSTRACT

A dental suction arrangement which removes saliva and other fluids from a person's oral cavity. The suction arrangement includes a suction tube and an absorption body to be placed in the oral cavity. The suction tube includes a suction part which is arranged in the absorption body and which is equipped with a hole organs or elements communicating with the absorption body. The suction arrangement is made of elastically deformable material in order to adapt to the anatomical shape of the bottom of the mouth.

### CROSS-REFERENCE TO RELATED APPLICATIONS

This application is a continuation-in-part application of PCT/EP2016/064618 filed on Jun. 23, 2016, which claims priority to Swedish patent application No. 1550893-0 filed on Jun. 26, 2015, and this application is also a continuation-in-part application of U.S. patent application Ser. No. 15/538,943, filed Jun. 23, 2016, which claims priority to EP 16205406.8, filed Dec. 12, 2016, all the disclosures of which are incorporated herein by reference in their entirety

### TECHNICAL FIELD

The invention is about a dental suction arrangement which removes saliva and other fluids from a person's oral cavity. The suction arrangement includes a suction tube and an absorption body to be placed in the oral cavity, thereby the suction tube includes a suction part which is arranged in the absorption body and which is equipped with a hole organ or hole elements communicating with the absorption body.

### BACKGROUND

In the dental field different types of suction arrangements are used for removal of saliva and water from the oral cavity. The supply of water mainly takes place when the tooth substance is drilled and grinded and when removing tartar. To cool the teeth, using water spray is very pleasant to the user, in order to lower the mouth temperature. Water supply is also common in connection with teeth—and oral cavity—cleaning where water spray is used. The removal of saliva and water is beneficial for several reasons, including patient's or users comfort, working area visibility, dry keeping of an operating area, The removal of saliva and water is required for several reasons for example user's comfort, to improve visibility in the cleaning process, and to keep the operating area dry.

The removal of saliva and water is mainly performed using two kinds of suction devices including tubes with different dimensions, where the thicker one is a so-called high-speed suction device that efficiently removes large volumes of water and saliva, and which is operated manually by the operator or his assistant. The suction device with a thinner dimension and lower suction capacity is mainly used in connection with a device which is fixed in the oral cavity and where the removal of saliva and water works automatically without impact of the operator. Often the suction device also works as a protection of the tongue against rotating instruments by keeping away the tongue.

One example of such a thinner-dimensioned suction device is described in U.S. Pat. No. 3,086,289. These kinds of suction arrangements consist solely of various kinds of plastic tubes, straight or pre-bent in different shapes, with holes placed in one end or along the tube. By reshaping the suction device by hand it is possible to adjust the device to different locations in the oral cavity where the absorption at the moment it is most needed and where the suction device brings most benefit.

The problem is that all people look different in combination with the fact that the constructions are rather static with limited possibilities for adjustments of the absorption effect to areas where it is most needed. These constructions are neither able to absorb saliva and water further down at the bottom of the mouth adjacent to the Glandula Sublingualis nor further back in the throat and they have only limited ability to safely isolate an operating area from saliva since the absorption effect is limited to the area where the suction device's holes are located.

Another major problem is the low comfort of these constructions. The need to remove saliva and water is often highest at the bottom of the mouth but when the suction device is placed there it almost always will be in contact with the inside of the lower jaw, which is sensitive to pain and it often results in a strong discomfort for the patient or user. To minimize this effect, now and then a need arises to adjust and change the shape of the suction arrangement, which takes time from the operators other work.

When placing the suction device at the bottom of the mouth, the suction device's holes often get blocked since the mobile part of the mucous membrane is absorbed into the holes and this leads to a removal or to a strong decrease of the device's absorption capacity. Additionally in such cases, often a loud and very disturbing noise arises when the absorbed mucous membrane vibrates in the suction device's holes.

From the past it is further known to provide plastic tubes with absorption bodies and place these along each side of the tooth socket or along the row of teeth. Accordingly, some of the above described negative effects and problems connected with saliva absorption can be reduced. Examples of such types of suction devices are described in U.S. Pat. No. 5,071,347 and U.S. Pat. No. 6,309,218, which are hereby incorporated by reference.

In order to efficiently absorb saliva and water from the oral cavity using such an absorption body it is important that the absorption body has good connection/coverage with the area where the saliva is produced and accumulated. The absorption body of the above-mentioned known suction arrangement is cylindrical. This results in an incomplete connection with the respective area. Certainly some of the absorption bodies have some elasticity which allows the absorption body to compress so that the area of the connection points can be expanded, however only limited. This leads to an unsatisfactory absorption, and is why the advantages in working only with absorption tubes are limited.

In US 2013/0203012 a siphoning device for surgery is described that has similar basic features but where the physical properties are very limited regarding dental use. The described device can not be placed in the desired area where it is most desired, between the tongue and the teeth row, in an easy way or in a secured fixed position. It is not protecting the tongue from a rotating instrument or a similar instrument at all.

All common suction devices are today one-sided which are very insufficient when there is a need for keeping both sides dry i. e. during the cementation of a full-arch bridge in the lower jaw where the dry-keeping is essential. This is an operation of great stress for the dentist and assistant. With this solution the device could stay in place creating the needed dry operation field and eliminating the risk of saliva contamination. Using two ordinary devices would not help then they have to be removed before placing the bridge and then the risk for contaminations is very high.

Another general problem with all different suction devices today are when there is time for i.e. checking and adjusting the height of a crown or a filling it is necessary for the patient to bite together on a occlusion-foil for having a colour-mark on occlusal bad spots. The suction device then has to be removed from the mouth. When the suction device is removed the saliva flows all over the teeth. The teeth have to be dried with the vacuum-suction device before the patient is biting on the occlusion-foil. If the tooth surface is wet the color from the occlusion-foil is not adhering so well to the tooth and the registration becomes false or unsure. The procedure with repeatedly drying is also very time consuming.

Another dry-keeping problem occur when using modern 3D-laser scanning techniques for taking so called digital impressions of the teeth and teeth-rows. They have to be saliva-free or else there will be a defect impression not possible to use for producing the prosthetic construction or similar. This is a problem especially with the lower jaw. When using today's suction devices where the suction tube is placed over the row of the teeth it will interfere with the recording when scanning a full row in the lower jaw.

A general problem in dentistry is also the noise-level that is generally high due to air-driven high-speed turbines, scalers and the suction devices. Then the suction devices normally is in action all of the time, especially the “low-volume devices” described here, they are very tiring for the operators staying in this environment all day long.

Also when placed for a long time in the mouth the soft tissue in the mouth will become over-dried resulting in discomfort for the patient.

These devices are very static and the absorbing bodies are placed along the row of the teeth in a fixed device, which is why absorption will only take place when the saliva/fluid reaches a certain level and comes in contact with the bodies. The area between the bodies are kept dry but all the remaining fluid and saliva will reach the throat of the patient as if there has not been any suction device at all.

Another disadvantage of these known cylinder-shaped absorption bodies is that they are placed along the tooth socket. When saliva accumulates in the oral cavity and rises to a level so that it tends to flow over in the patient's throat then the patient reflexively will swallow, and this swallowing movement will increase the level of saliva and water.

There exists a need of a effective suction arrangement which removes the saliva and water where it is produced and accumulated, that it shields off the operation field bilaterally and keeps it dry, is comfortable for the patient by not creating pain or an over-dried mouth, should be possible to let stay in the mouth during biting or without interfering a 3D-scanning procedure and is an intelligent system communicating with the vacuum-producing system in creating a low-noise environment and saving energy.

There exists a need for a suction arrangement which removes the saliva and water where it accumulates, i.e. at the bottom of the mouth, so that the operating area is kept dry and water-free and the patient's need to swallow are minimized, and especially do not cause discomfort for the patient when the suction arrangement comes in contact with the inside of the lower jaw.

### SUMMARY OF THE INVENTION

One object of the present invention is to remove the problems associated with the prior known technique in the field and thus provide a more effective absorption of saliva and other fluids than previously offered.

This and other objects are achieved, either completely or partially, from a dental suction device which removes saliva and other fluids from a person's oral cavity, where the suction arrangement includes a suction line and an absorption body to be placed in the oral cavity, where the suction line comprises a suction part which is placed in the absorption body and which is equipped with hole members communicating with the absorption body. According to the invention, the suction arrangement is made of elastically deformable material in order to adapt to the anatomical shape of the bottom of the mouth.

Through the elastically deformable material, the main part of the outer surface of the suction arrangement will be connected with areas at the bottom of the mouth from where the fluid will be absorbed. This enables a more effective and complete absorption.

Also, it is important that the suction line with the absorption body extends not only to the lower part of the lower jaw but also extends along the lower part of the lower jaw. By this, the absorption body is sort of clamped between the tongue and the lower jaw, such that it will not unintentionally disengage from this place.

This is especially important when the patient swallows since with the prior art devices, due to intense movements of the oral cavity, the suction tube usually gets lost.

The suction arrangement, which comprises elastically deformable material, adapts to the bottom of the mouth and covers the area without causing any discomfort for the patient and at the same time preventing the saliva from coming in contact with the teeth, and further the device is retained at the bottom of the mouth since it is partially placed under the tongue. The suction line may be deformed as desired to be adapted to the best position. It may extend over the molars or incisors to then extend close to and along the lower jaw in the distal direction. Preferably, it reaches the lower part of the lower jaw, i.e. at a level below the main tongue extension close to the canine, and then extends under the tongue along the premolars and molars.

The suction line is preferably made of plastic material which is easily and plastically deformable. The absorption body may be fixed to the suction part of the suction line in any suitable manner, such as by glueing or by friction. The suction arrangement is suitable for applying to both the left and/or the right lower jaw.

The absorption body within the suction arrangement also works as a pump when it is compressed due to movement when the patient swallows and the subsequent expansion. In order to ensure the desired and beneficial pump action, it is essential for the elastic absorption body to have a compressibility that is half of its original volume by standard tongue force. The compressibility may be considerably higher, i.e. up to 85 percent. Thus, the material of the absorption body may be soft open foam with a low elasticity coefficient, or a soft flocking, or a silky mesh covering the inner absorption body.

The absorption body also eliminates the need to interrupt e.g. the ongoing dental cleaning treatment for adjustment of the suction device when it causes painful pressure to the patient's inside lower jaw, which is very pain sensitive.

Due to the fact that the suction arrangement adapts itself to the anatomical shape of the bottom of the mouth and also is elastic, it automatically very efficiently adapts to the exact anatomical shape in a way which is not possible with a simple cylindrical absorption body under known techniques. Also the dental treatment will be more silent since the absorption body acts as a noise shield, dampening the suction noise emitted by suction holes. The created anatomical form effectively allow the reception of saliva leaving both Glandula Sublingualis and Glandula Submandubularis, and the device builds a barrier between the teeth and the saliva glands.

In one embodiment of the invention, the absorption body shows, in a cross-sectional view, perpendicular to the length extension of the absorption body, a non-circular contour.

In another embodiment, the absorption body shows, in a cross-sectional view perpendicular to the absorption body's length extension, a contour with a base and a top, where the mentioned base is broader than the mentioned top. This shape facilitates the placement and retention under a part of the tongue. In another embodiment, the absorption body has a part, which, in a cross sectional view perpendicular to the absorption body's length extension, shows a contour with at least one concave part. With the concave contour part the shape of the absorption body is adapted in a natural way in order to create space for the tongue. This will avoid concentration of pressure at the tongue and at the same time the absorption body effectively reaches areas, over as well as under the tongue, where the absorption is most needed.

Preferably, the cross-section of the absorption body is larger below the area of the waist which is created by the tongue and slightly smaller above this waist. The suction part of the suction tube is preferably arranged at the lower, broader part. In one embodiment, the suction tube is made of elastically deformable and adjustable material in order to obtain an adaption to the anatomical shape of the bottom of the mouth and placement position. This embodiment includes a variety of application possibilities. The suction device and its related suction tube or suction line and absorption body especially can adapt itself to different sizes of the bottom of the mouth, but also since the absorption body is so long it has the possibility to extend along the alveolar area or gingival area of the jaw at the incisors and canines as well as at premolars and molars, and due to the cross-sectional shape it adapts to the different parts.

In a further embodiment of the invention, the suction device includes an extension part which, when placed in the oral cavity, extends backwards towards the throat, whereby the extension part preferably has a reduced cross-sectional area which decreases in the direction of the throat. The extension part reaches a bit down in the throat which helps reduce the accumulation of fluid in the throat, which otherwise may cause discomfort and disturb the dental treatment due to swallowing reflexes. The extension part generally entails a lower fluid level.

In a further embodiment, the absorption body is adjustable in its length. A long absorption body will be prefabricated, and it will be shortened by cutting it. Alternatively, the absorption body may be fixed by friction on the suction part of the suction line, and be manually extended and shifted according to the needs. Further alternatively, the suction part may be provided with a punch pattern, allowing its extension together with the absorption body.

It is advantageous to have the U-shaped absorption body and U-shaped suction part at a suitable lengths which would fit into the largest jaws. When tweeting a patient with a smaller jaw, the suction part and the absorption body is shortened, i. e. by cutting them off. The free end hole of the suction part may be closed in any suitable manner.

In a further embodiment, the hole member comprises multiple holes. This facilitates the transport of fluid from the outer surface of the absorption body to the absorption part or suction part since the transport routes become shorter. Preferably, the multiple holes are distributed along the absorption part as well at its circumference.

In a further embodiment, the holes are of different size and/or shape. The hole size and/or shape or both can thus be adjusted to local variations in relation to the need of absorption at the different points of the absorption body. The need of absorption may vary depending on where the saliva is produced, on locations where saliva tends to accumulate, on locations where other fluids accumulate and on variations in the absorption body's deformation. Also from a flow technical point of view it is favorable to be able to have different sized holes depending on their distance to the connection of the absorptions part to the rest of the suction device.

In a further embodiment, the suction device includes a secondary absorption body to be placed at the Glandula Parotis excretory duct, where the secondary absorption body is in fluid connection with the absorption body.

A substantial part of saliva is produced by the Glandula Parotis excretory duct, i.e. a salivary gland located in the back upper cheek area. By arranging an absorption body even in this area, it is possible to absorb the saliva already at the source where it is produced and it is not necessary to take care of it when it accumulates into the bottom of the oral cavity. Additionally, it also prevents the saliva from the Glandula Parotis from reaching the teeth in the upper jaw's side areas, which is beneficial and comfortable to the patient. This results in an increased efficiency of the suction device.

In a further embodiment, the secondary absorption body and the absorption body are connected with each other via a connecting line, which can be called the secondary suction line. It would also be possible to achieve the transport by capillary action through an extension of the absorption body to the secondary absorption body. However, the flow becomes more efficient if performed through a line between both bodies.

In a further embodiment, the suction device includes multiple absorption bodies and the suction tube includes additional lines, whereby each additional line is connected with a suction part in the respective absorption body.

In this embodiment, the secondary suction line may be formed such that a spring force is exerted between the secondary absorption body and the primary absorption body. By this, both are kept in place, and the primary absorption body is pushed down to be closer to Glandula Lingualis.

This embodiment allows for a simultaneous fluid removal from several locations in the oral cavity with just one suction device. The user can decide freely whether absorption bodies should be placed on two or several different points on the right side between the tongue and the row of teeth, on the right side between the row of teeth and cheek, correspondingly on the left side, between the row of teeth and lip, at the Glandula Parotis or on other points in the oral cavity.

In a further embodiment, the respective suction part is detachably connected with the rest of the suction line.

This allows for an easy replacement of absorption bodies if, for some reason, the absorption capacity declines. This also allows for one and the same suction tube to be connected to different types of absorption bodies.

In a further embodiment, the suction device includes a suction line and a set of multiple absorption bodies in different embodiments with respect to the anatomical shape and/or size.

With such a set, a high flexibility is achieved, since a single suction line can be connected to any type of absorption body depending on the current situation. Alternatively, it allows having a set of one absorption body-type in different sizes to choose between for an optimal fit depending on the patient's jaw size or other patient-dependent variables.

In a further embodiment, the suction device includes a protection shield, which may be a tongue protection in order to block the tongue. The tongue protection aims to protect the tongue from rotating instruments during the dental treatment such as a cleaning treatment, and also to prevent the tongue from touching the tooth or teeth in critical areas which may cause a contamination with saliva when performing different filling and/or cementation of prosthetic constructions.

In one embodiment, the tongue protection is arranged at an extension of the suction line, and in another embodiment, the tongue protection is arranged at one sidearm of the mentioned suction line.

In a further embodiment, the tongue protection is made of a material which is elastically deformable. With this embodiment, the advantage provided is that the tongue protection can be bent in order to effectively separate the tongue from the jaw area, while also allowing the assisting personnel to obtain full access and insight into any critical area for required interventions and complementary removal of water and saliva.

In one embodiment, the tongue protection is made of a material which can be broken off in order to adjust the fitting and placement in the oral cavity and also in order to enable an increased access and insight, while the ability to protect the tongue is retained.

In a further embodiment, the tongue protection is adjustably arranged to the suction tube. This provides an additional opportunity to perform specific adjustments depending on the condition of the patient.

In a further embodiment, the tongue protection has an absorption capacity. This absorption capacity can either be provided in the case that the tongue protection is made of absorbable material, or covered by absorption material or in the case that the tongue protection is equipped with a secondary suction line which is connected with the suction device and its related absorption body.

In one embodiment, the respective tongue protection and suction part are detachably connected with the rest of the suction tube.

The secondary suction line between the tongue protection and the absorption body may be such that it is biased to separate the tongue from the lower part of the lower jaw. By this, the absorption body is pressed downward towards the Glandulae under the tongue. The secondary suction line advantageously is bent by about a little less than 180 degrees.

In another embodiment of the invention, the tongue holder may be provided with a grip, like a small lateral extension. By this, any operator like a dentist may pull the tongue holder sidewards or in any suitable direction.

In another embodiment of the invention, the tongue holder is turnable and/or attached via a plastic hinge to the secondary suction line.

In another embodiment of the invention, the tongue holder has a structured surface. This is for the tongue, which tends to like some activity, to play with it.

In another embodiment of the invention, the tongue holder is made from thermosetting material. The operator then may heat it before use, and shape it to any desirable form. After re-cooling, it will maintain the selected form. This process may be repeated frequently if desired.

The tongue holder advantageously may be made from orange acrylic material to form a light curing shield.

The suction line may be made of any suitable material such as special plastic material. The material may be selected to have a memory effect, and/or may be plastically and/or elastically deformable.

In one embodiment, the suction device includes a suction tube or suction line and a set of multiple absorption bodies in different executions with respect to the anatomical shape and/or size, and a set of multiple tongue protections in different executions with respect to the anatomical shape and/or size.

In a very advantageous embodiment of the invention, the absorption body is covered by a silky mesh, such as known from jewelry boxes. Such a web or mesh has a surface with a low friction coefficient such that the absorption body may be inserted easily to its desired place. It has a very pleasant and comfortable feeling for the patient. Yet, its permeability is sufficient to have the desired suction force retained therethrough.

Preferably, the silky mesh is very thin and thus very flexible. Also, the absorption body is soft and flexible and has an elastic character. If it is made from foam or any other suitable material, a hardness gradient may be used such that it is harder close to the suction part and softer close to its outer periphery.

Such a hardness gradient automatically is provided if the absorption body is made of a flocking. Any desired shape and distribution is possible. Flocking has a very good capillary effect.

In another embodiment, the suction arrangement is provided with a moisture sensor, intended to give a feedback to the vacuum pump and to reduce the pump action if the area under the tongue is dry. This will also reduce the vacuum pump noise.

The inventive suction arrangement is also very suitable for unteethed patients. Because of the spring action both to the absorption body between the tongue and the bottom of the mouth, and of the suction line between the chin and the lower part of the lower jaw, the invention works great independently of whether the jaws are fully teethed, partially teethed or unteethed.

In a further embodiment, the absorption body and the suction part may be essentially U-shaped for covering the full arch of the lower jaw. The suction-line is connected to a vacuum source and the suction line exits from the absorption body at one end of the absorption body. The other end may be covered by the absorption body, may be provided with a hole or may be closed, depending on the needs. The absorption body and the suction part, in an advantageous development, are plastic, elastic and/or plastically deformable. Thus, they may be deformed according to the desired shape.

The absorption body and the suction part may be arranged oraly close to the lower jaw. The suction line when exiting the absorption body is bent upwardly to pass over the lower jaw. This portion of the suction line, the transverse portion, is adapted to transverse the lower jaw at a tooths free area thereof, one sided or bilaterally. Following this transverse portion, the suction line is intended to run further exiting the mouth of the patient.

Thus, the dental suction arrangement according to the invention may be effective also during occlusion.

Having the suction device in place in the mouth gives great advantages as it is saving a lot of time in reducing time for dry-keeping because this procedure some times needs to be repeated several times before a perfect result is achieved. The teeth are kept dry and the colour from the occlusion-foils are easily attached to the dry surface. Also grinding the specific tooth is much more convenient when it is not covered with saliva or water.

With the suggested design the suction device will stay in place creating a dry scanning-field without any interfering parts from the suction device.

With the suggested design the noise is reduced but besides the physical properties reducing the noise-level a built-in moisture-sensor or flow meter is suggested. The moisture-sensor could be placed in the foam-body or on the suction-tube. Preferable of a wireless type sending information to the suction system to reduce the “power” needed in the system. This will lower the surrounding tiring noise level and reduce the fatigue feelings created by the high noise levels then the system will only operate on high levels when needed.

Other purposes, features and facilities of the invention will be shown in the following detailed description, in the patent claims, as well as in the drawings. It should understood that additional advantageous embodiments may arise via different possible combination of the features from the described embodiments and with each possible combination of these features described in the examples following presentation.

Generally, the terms in the requirements should be interpreted in accordance with their normal meaning in the technical field, unless explicitly stated otherwise. All references to “a/an/the [a suction arrangement, a suction tube, an absorption, etc.]” should be interpreted openly as a reference to the existence of at least one mentioned suction device, suction tube, absorption body etc. unless explicitly stated otherwise.

### BRIEF DESCRIPTION OF THE DRAWINGS

These and other embodiments of the invention will now be described in more detail with reference to the attached drawings, which show embodiments of the invention.

FIG. 1 is a side view of a suction device or suction arrangement according to a first embodiment of the invention.

FIG. 2 is a longitudinal section through the suction device in FIG. 1.

FIG. 3 is a sectional view along line III-III in FIG. 2.

FIG. 4 is a sectional view corresponding to the view shown in FIG. 2 illustrating the fluid flow.

FIG. 5 is a sectional view corresponding to the view shown in FIG. 3 illustrating the fluid flow.

FIG. 6 is a top view of the suction device shown in FIG. 1 placed at the bottom of the mouth between the lower jaw's alveolar area and the tongue.

FIG. 7 is a sectional view along line VII-VII in FIG. 6.

FIG. 8 is a top view of the suction device shown in FIG. 1 placed at the bottom of the mouth in a displaced position in relation to FIG. 6.

FIG. 9 illustrates a detail of the suction device according to one embodiment of the invention.

FIGS. 10 through 14 illustrate sectional views through a detail of the suction device according to five different embodiments of the invention.

FIG. 15 is a side view of a further embodiment of a suction device according to the invention.

FIG. 16 is a top view of the suction device shown in FIG. 15.

FIG. 17 is a side view of a further embodiment of a suction device according to the invention.

FIG. 18 illustrates a detail of the embodiment shown in FIG. 17.

FIG. 19 shows a sectional view of another embodiment of the suction arrangement of the present invention, with a modified absorption body.

FIG. 20 shows another embodiment of the suction arrangement according to the invention.

FIG. 21 shows still another embodiment of the suction arrangement according to the invention.

FIG. 22 is a perspective view of a suction device or suction arrangement according to an embodiment of the invention.

FIG. 23 is another embodiment of the suction device or suction arrangement according to the invention.

FIG. 24 is another embodiment of the suction device or suction arrangement according to the invention.

FIG. 25 is a sectional view along line III in FIG. 23.

FIG. 26 is a perspective view of another embodiment of the invention.

FIG. 27 is a perspective view of another embodiment of the invention.

FIG. 28 is the embodiment according to FIG. 27 in a cross-sectional view.

FIG. 29 is a perspective view of another embodiment of the present invention.

FIG. 30 is a perspective view of another embodiment of the present invention.

FIG. 31 is a perspective view of another embodiment of the present invention.

FIG. 32 is a perspective view of another embodiment of the present invention.

FIG. 33 is a perspective view of another embodiment of the present invention.

FIG. 34 is a perspective view of another embodiment of the present invention.

FIG. 35 is a perspective view of another embodiment of the present invention showing the extension parts of the U-shaped body.

FIG. 36 is a perspective view of another embodiment of the present invention with built in moisture sensors/flow meters with wire connection.

FIG. 37 is a perspective view of another embodiment of the present invention with built in moisture sensors/flow meters with wire-less transmitting.

FIG. 38 is a perspective view of another embodiment of the present invention.

FIG. 39 is a sectional view of another embodiment of the suction arrangement of the present invention, with a modified absorption.

FIG. 40 is still another embodiment of the suction arrangement according to the invention.

FIG. 41 is a perspective view of another embodiment of the present invention.

FIG. 42 is a perspective view of another embodiment of the present invention.

FIG. 43 a partial sectional view according to FIG. 41.

FIG. 44 is a sectional view of another embodiment of the present invention.

### DETAILED DESCRIPTION

The invention will now be described in more detail with reference to the attached drawings. Like parts in different embodiments may be described using the same reference numerals.

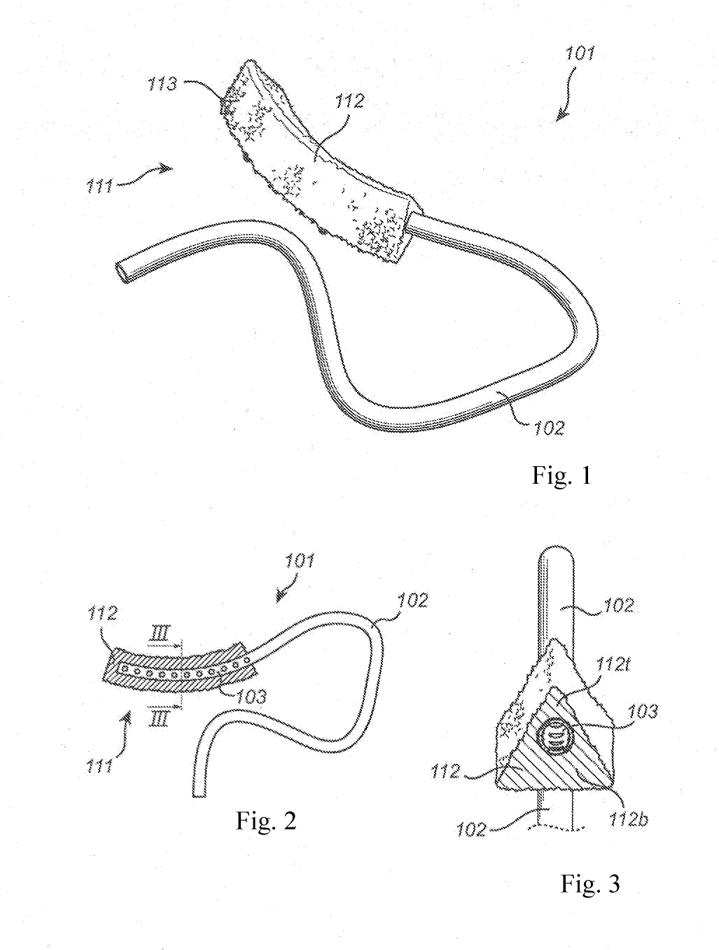

In FIGS. 1 to 8, a first embodiment of the invention is illustrated. As shown in FIG. 1, the suction device has a suction tube or suction line 101 which can be connected to a vacuum source in order to create a negative pressure inside the suction tube. The suction tube 101 has an exposed part 102 which is visible. The suction tube 101 extends into the absorption body 111 of the suction device with a suction part 103 embedded in the absorption body (not visible in FIG. 1). The absorption body 111 consists of a main part 112, and optionally of an extension part 113. The main part 112 is arranged to be placed at the user's bottom of the mouth between the lower part of the lower jaw and the tongue.

The extension part 113 is arranged to extend backward from the main part 112 towards the patient's throat. The exposed part 102 of the suction tube is conventionally bent to easily be fixed around the patient's chin. The suction device is shown in FIG. 2 in a sectional view of its length extension whereby suction part 103 of the suction tube 101 is visible. In this example, exposed part 102 of the suction tube is shown. The suction part 103 may alternatively be jointed to the juncture between the suction part 103 and the exposed part 102. The suction part 103 is equipped with multiple holes 104 which connect the tubes inner part with the covering absorption body. In this example, the absorption body is smoothly arch-shaped, but since the suction device is made of elastic deformable material the device can adapt to other shapes in order to fit the patient's oral cavity and the bottom of the mouth, in particular.

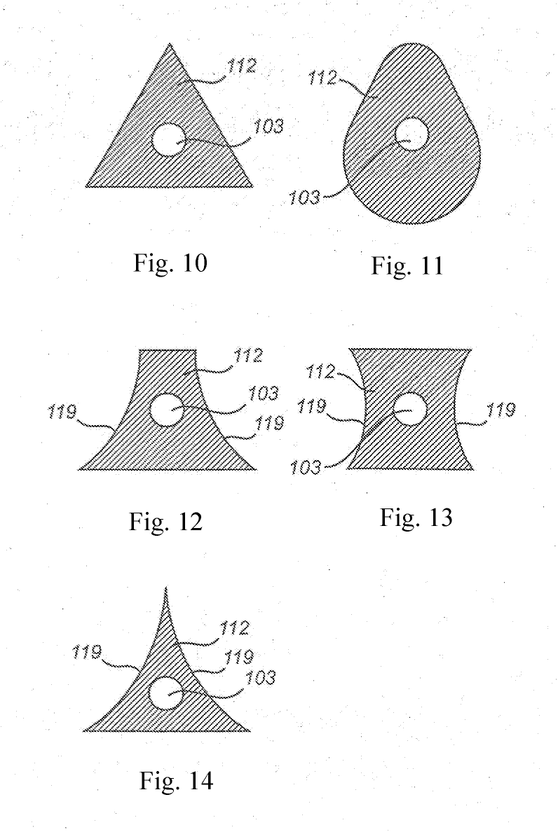

FIG. 3, which is a sectional view along line III-III in FIG. 2, shows that the absorption body in the lateral direction has a non-circular outer contour with a base 112b and a top 112t, whereby the base 112b is broader than the top 112t, and shows a triangular-like cross section. The suction part 103 of the suction tube 101 is centrally located in the absorption body. Alternatively to the illustration, this part can be located in other positions in the absorption body, e.g. in an asymmetric placement. Also, the outer contour may be different than the exemplified. Examples of different contours are shown in FIGS. 10 to 14.

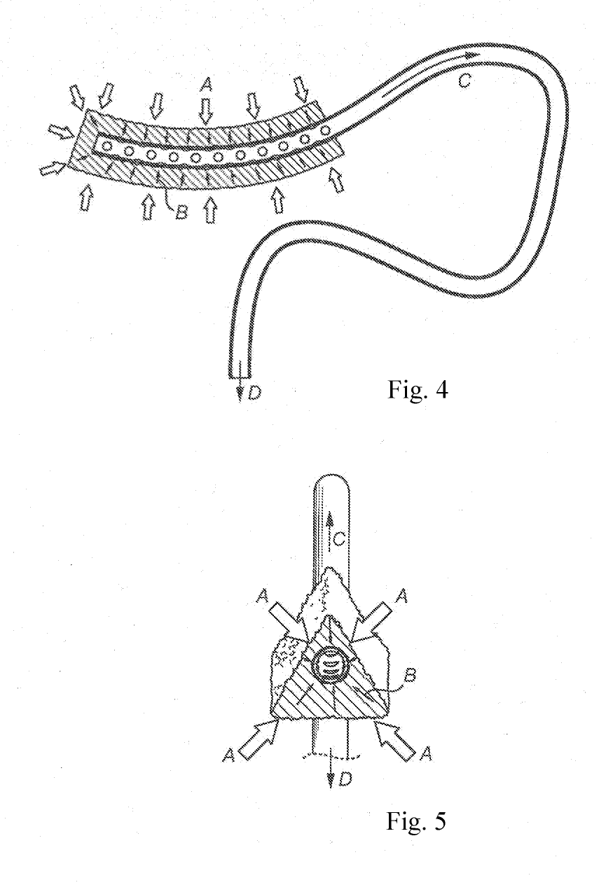

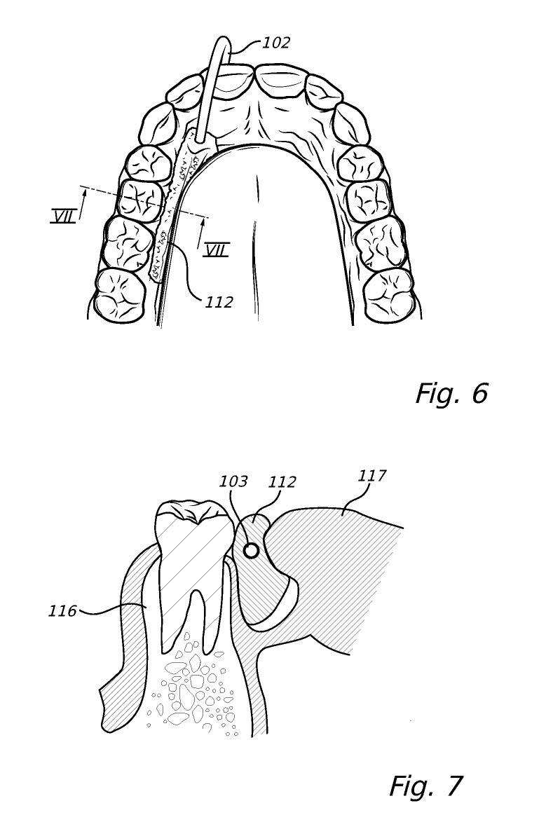

FIGS. 4 and 5 schematically show the fluid flow through the suction device. Saliva and water enter the absorption body 111 from mucous membranes and from other places in the oral cavity, where the fluid accumulates, as indicated by arrows A. The suction tube 101 is connected to vacuum whereby underpressure is generated inside its suction part 103. By capillary action the fluid entering the absorption body 111 is absorbed in the direction of the suction part 103, as indicated by arrows B. Through the vacuum the fluid is absorbed by the suction tube according to the arrow C, and is removed according to arrow D. FIGS. 6 and 7 illustrate the suction device in place at the bottom of the mouth between the lower part of the lower jaw 116 and the tongue 117.

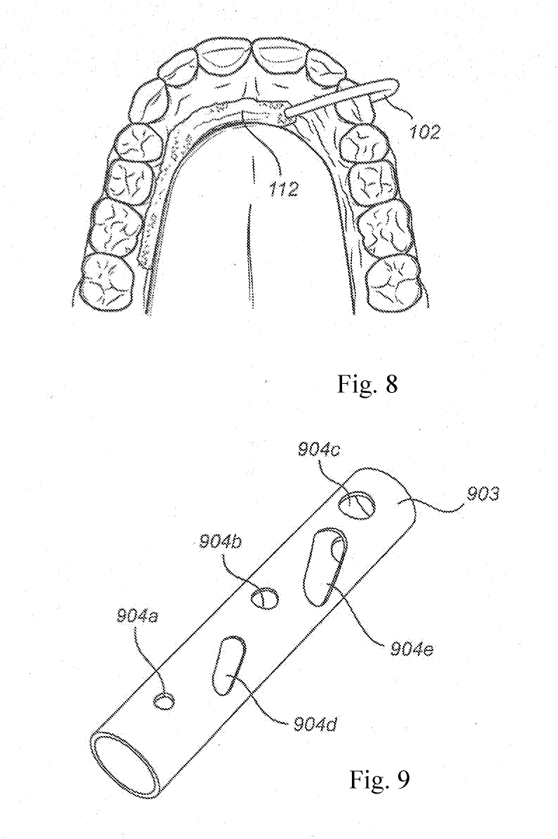

FIG. 8 illustrates the suction device placed in a slightly displaced position in relation to FIG. 6. This positioning can be beneficial in filling and cementation of prosthetic constructions in the front part of the patient's mouth. Since the suction device is made of elastic deformable and adjustable material, the suction device can be bent and be placed at the bottom of the mouth beside the lower part of the lower jaw by the incisors and extends back toward the lower part of the lower jaw by the premolars and molars on the right and/or left side.

The suction tube 101 is preferably made of extruded plastic and if needed also includes an integrated wire that allows the operator to reshape the tube if necessary. The suction tube has a prefabricated shape and size that allows it to be easily placed at the bottom of the mouth and at the same time to cover the lower part of the lower jaw for a secure fixation.

The suction tube may have a round shape or an oval shape, which is even more easy to bend. Also, the wire may be flat, having a softer spring characteristic in its flat direction. Both may be adapted to the users needs.

The suction part 903 of the suction tube can, as illustrated in FIG. 9, be provided with holes of different size and shape. The holes 904a, 904b, and 904c are circular with different diameters where the holes 904a closest to the exposed part of the suction tube or line portion are the smallest. The holes 904d and 904e are elongated.

The absorption body 111 is made of suitably elastically deformable material which enables fluid transport through the body, preferably foam plastic with open cells. It is also possible to use other soft materials that have capillary action like, for example, cotton fibers, various textile fibers, synthetic fibers, mull, lint or gauze, preferably covered by a mesh, and the like.

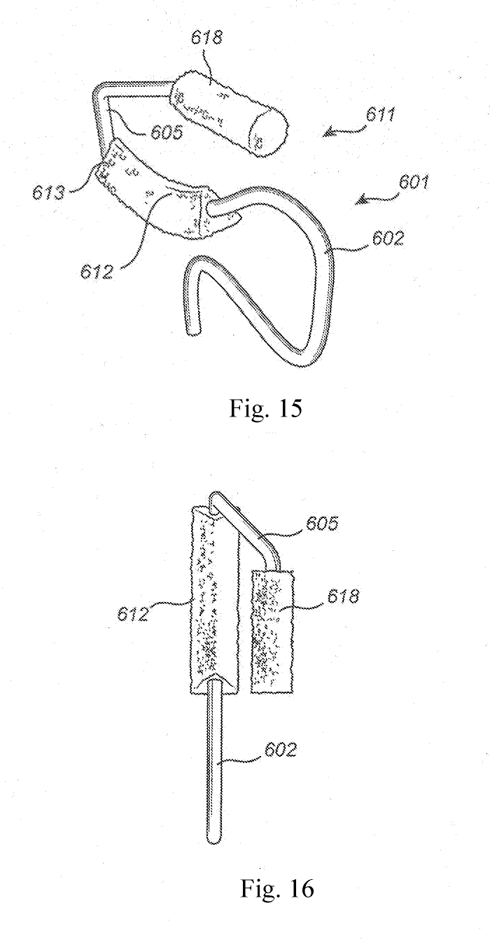

In the embodiment example illustrated in FIGS. 15 and 16, the suction device includes a secondary absorption body 618 beyond the main absorption body 612, 613. The secondary absorption body 618 is arranged to be placed at the Glandula Parotis excretory duct and is connected with the main absorption body via a connecting line 605. The main absorption body is arranged for an application as illustrated in FIG. 6. Via the secondary absorption body 618, saliva from the Glandula Parotis is directly absorbed where it is generated and via the main absorption body transferred to the suction tube 602. The connecting line 605 can advantageously be used as an extension of the suction part of the suction tube, but an arrangement on an additional line of the exposed part 102 of the suction tube is also possible or an arrangement as an additional line to the suction part 103 before the transition to an extension part.

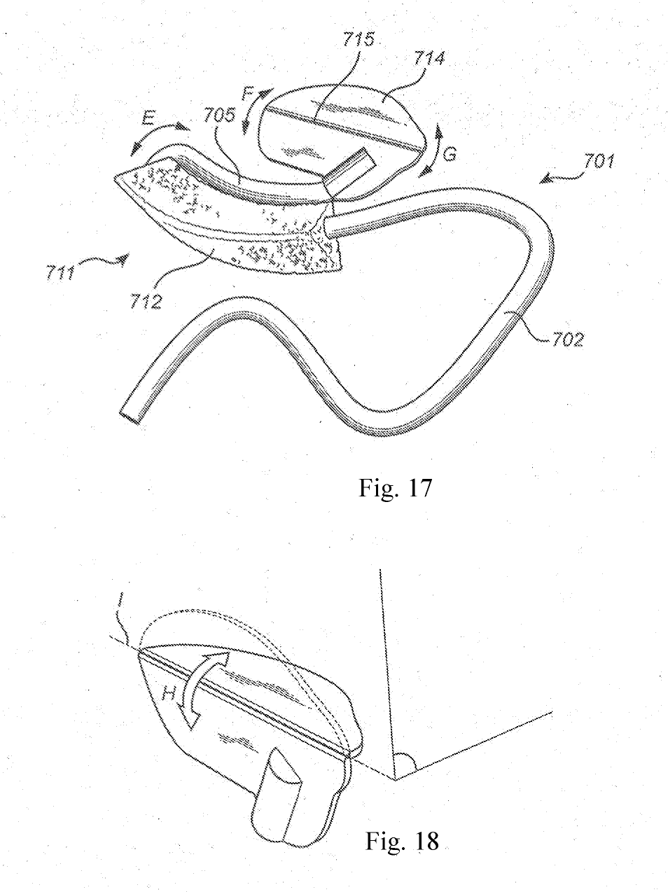

In the embodiment example illustrated in FIG. 17, the suction device comprises a tongue protection 714. The tongue protection aims to block the tongue and so protect the tongue from rotating instruments during the dental treatment and also to prevent the tongue from touching the tooth or teeth in any critical area which may cause a contamination with saliva when performing different filling and/or cementation of prosthetic constructions. FIG. 17 illustrates an embodiment, where the tongue protection 714 is arranged in an extension 705 of the suction tube 702, but in another (not illustrated) embodiment, the tongue protection may be arranged on one sidearm of the exposed suction tube 702. In a further (not illustrated) embodiment, the suction device includes two tongue protections, one arranged as illustrated in FIG. 17, and one arranged on one sidearm of the exposed suction tube 702. The tongue protection can be made of elastically deformable material. Thus, the tongue protection can be bent, as illustrated with arrow H in FIG. 18, along line I, and therefore effectively separates the tongue from the operation, while also allowing the assisting personnel to obtain full access and insight for required interventions and complementary removal of water and saliva.

In one embodiment, the tongue protection is made of a material which can be broken off, e.g. along line 715, in order to adjust the fitting and placement in the oral cavity and also in order to enable an increased access and insight while the ability to protect the tongue during treatment is retained.

Furthermore, the tongue protection may be arranged in relation to the suction tube's 702 suction part 703 and the absorption body 712, as illustrated by arrows E, F and G in FIG. 17.

In a further (not illustrated) embodiment, the tongue protection has an absorption capacity. This absorption capacity can either be provided in the case that the tongue protection is made of absorbable material or when the tongue protection is equipped with a suction tube which is connected with the suction device and its absorption body.

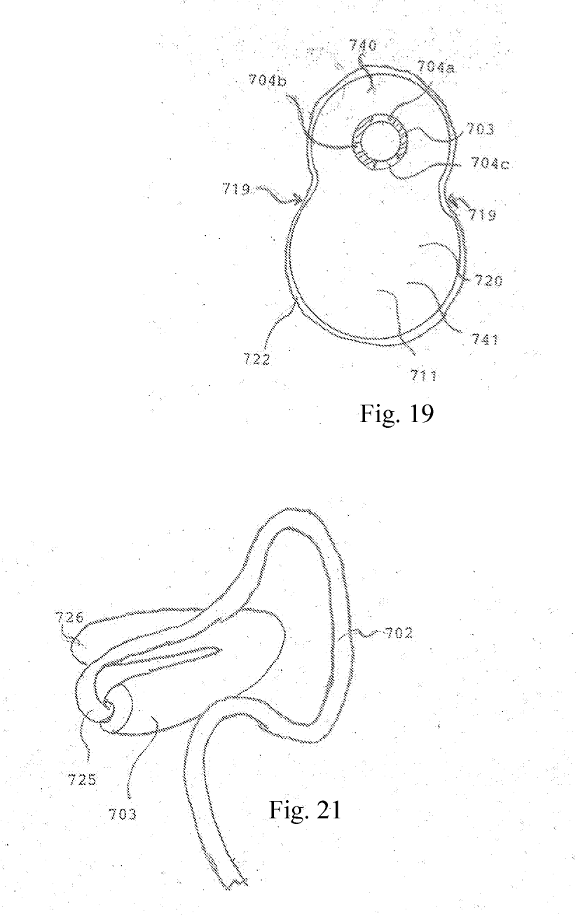

FIG. 19 shows another embodiment of the invention. According to this embodiment, the absorption body 711 is formed such that it is noncircular, with a base and a top, whereby the base 741 is broader than the top 740. The general form corresponds to an egg, however with a waist such that having concave parts 719 separating the top portion 740 and the base portion 741.

According to this embodiment, a huge suction buffer is provided which is intended to at least partially fill out the space under the patient's tongue. Also, with such a huge buffer, the desired pump action created by the force exerted by the tongue is improved.

The absorption body 711 is made by an open-porous foam with a high elasticity, i. e. a soft open foam. This foam, according to the shown embodiment, is covered by a silky mesh which is also open-porous, to let the fluid pass, and is pleasant and comfortable to the patient. Alternatively, it can be closed-porous to block fluids.

FIG. 21 shows another embodiment of the inventive dental suction arrangement.

A suction part 703 passes through the top portion 740. It has a plurality of suction holes 704a, 704b and 704c which are distributed unevenly and with different diameters and forms, all over suction part 703.

Preferably, the suction part 703 extends in the center of the top portion 740. In a different embodiment it may extend eccentrically, in order to increase the suction power at one side of absorption body 711.

According to this embodiment, a tongue holder 714 is arranged at the end of a second suction line 705. The absorption body 711 is arranged between the second suction line 705 and first suction line 702. It comprises a suction part 703 with a plurality of suction holes 704.

As may be taken from the arrows C, D and E, all parts of this embodiment may be easily adjusted and bent according to the needs. Generally, it is preferred to have the second suction line 705 being bent by about 150 to 180 degrees and also the first suction line 702 bent several times.

The idea is to have the absorption body 703 being arranged as low as possible under the patient's tongue in the bottom of the mouth, close to glandolar submandibularis and glandouar sublingualis, and the tongue holder 711 extending upwardly from there and pressing the tongue obliquely upwardly and away from the jaw.

The first suction line 702, on the other hand, will cross over the canines or incisors of the patient and leave the mouth to be attached to a suction source like a vacuum pump.

By such an arrangement, both the absorption body 703 is pressed down, and the tongue holder 711 is pressed upwardly. Thus, it is preferred to have a certain section in the second suction line which should be bendable, in order to adapted to the patient's needs.

Another embodiment is shown in FIG. 21. This embodiment comprises a suction line 702 and a so-called “full jaw” absorption body 703. This absorption body is essentially shaped-shaped and comprises a suction part with suction holes even though this is not shown in FIG. 21. It is intended to be placed under the tongue and the suction line 702 is of a flexibility such that it may be bent at a hinge area 725, to have this essentially U-shaped absorption body inserted into the oral cavity either such that the suction line 702 is arranged on the left side or on the right side.

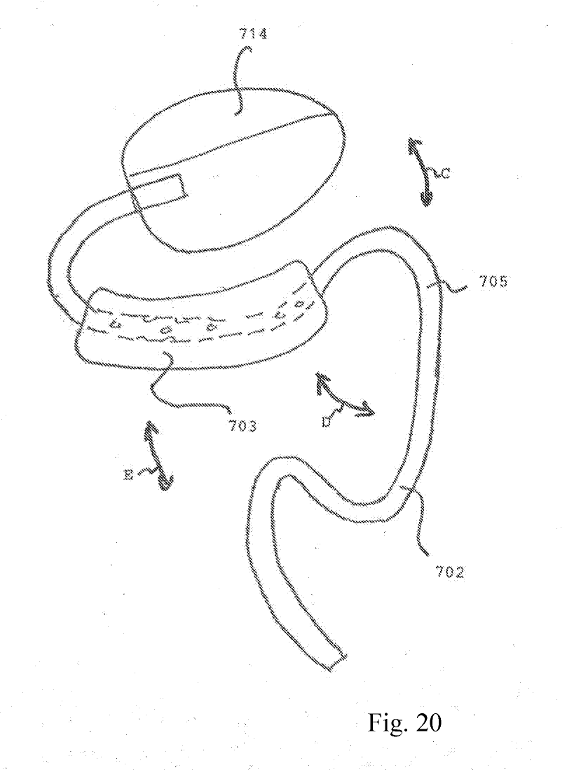

In another embodiment which is not shown, an additional tongue holder is arranged at the end 726 of the absorption body 703. The form thereof may be similar to the form shown in FIG. 20, and a second suction line may be provided between the tongue holder 714 and the end 726.

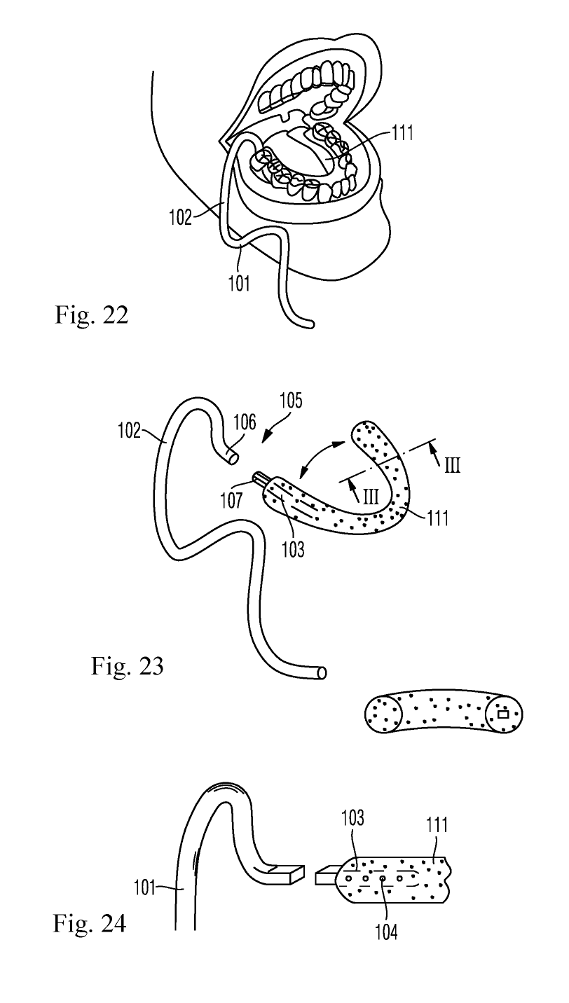

In FIG. 22, another embodiment of the invention is illustrated. As shown in FIG. 22, the suction device has a suction tube or suction line 101 which can be connected to a vacuum source in order to create a negative pressure inside the suction tube. The suction tube 101 has an exposed part 102 which is visible. The suction tube 101 extends into the absorption body 111 of the suction device with a suction part 103 embedded in the absorption body (not visible in FIG. 22). The absorption body 111 which surrounds the suction part 103 is arranged to be placed at the user's bottom of the mouth between the lower part of the lower jaw and the tongue.

The absorption body 111 thus follows the oral side of the dental arch. The absorption body is essentially U-shaped, or, to be more specific, parabolic. The form of the absorption body 111 is mainly determined by being adjacent or close-fitting to the lower part of the lower jaw. On the other hand, the suction part 103 extends through the absorption body 111. The suction part 103 has a greater stiffness than the absorption body 111 which absorption body 111 comprises preferably an open porous foam which, in an advantageous development, is covered by a silky mesh. The suction tube or suction line 101 which also comprises the suction part 103 is made from deformable plastic, and the suction part 103 comprises holes through which saliva or similar fluids may be sucked away.

In this example, the exposed part 102 of the suction tube 101 is shown. The suction part 103 may alternatively be joined to a juncture 105 between the suction part 103 and the exposed part 102. This is shown in FIGS. 23 and 24. The junction 105 comprises a sleeve 106 formed on the exposed part 102 and a plug 107 formed on the suction part 103.

The suction part 103 is equipped with multiple holes 104 which connect the tubes inner part with the covering absorption body. In this example, the absorptions body is arch-shaped or parabolic shaped, but since the suction device is made of elastic deformable material the device is adapted to fit into the patient's oral cavity and the bottom of the mouth in particular.

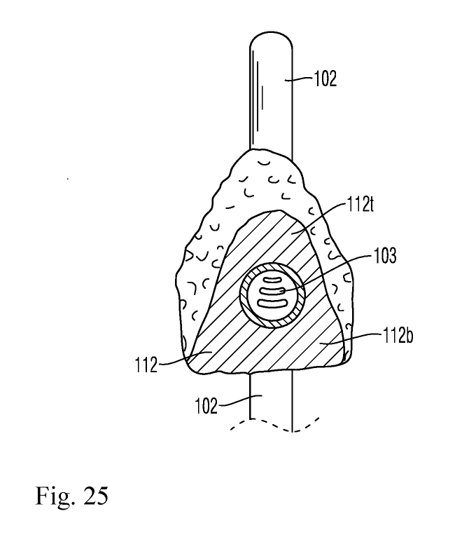

FIG. 25, which is a sectional view along line III-III in FIG. 23, shows that the absorption body in the lateral direction has a non-circular outer contour with a base 112b and a top 112t, whereby the base 112b is broader than the top 112t, and shows a triangular-like cross section. The suction part 103 of the suction tube 101 is centrally located in the absorption body. Alternatively to the illustration, this part can be located in other positions in the absorption body, e.g. in an asymmetric placement. Also, the outer contour may be different than the exemplified.

The suction line 101 is connected to a vacuum source which is not shown. Based on this vacuum, there is a fluid flow through the suction device 101. Saliva and water enter the absorption body 111 from mucous membranes and from other places in the oral cavity, where the fluid accumulates. The suction tube 101 is connected to vacuum whereby underpressure is generated inside its suction part 103. By capillary action the fluid entering the absorption body 111 is absorbed in the direction of the suction part 103. Through the vacuum the fluid is absorbed by the suction tube, and is removed.

Since the suction device 101 is made of elastic deformable and adjustable material, the suction device can be bent and be placed at the bottom of the mouth beside and along the lower part of the lower jaw by the incisors and extend back toward the lower part of the lower jaw by the premolars and molars on the right and/or left side.

The suction tube 101 is preferably made of extruded plastic and if needed also includes an integrated wire that allows the operator to reshape the tube if necessary. The suction tube has prefabricated shape and size that allows it to be easily placed at the bottom of the mouth and at the same time to cover the lower part of the lower jaw for a secure fixation.

The suction tube may have—in a cross-sectional view—a round shape or an oval shape which is even more easy to bend. Also, the wire may be flat, having a softer spring characteristic in its flat direction. Both may be adapted to the users needs.

The suction part 103 of the suction tube can, be provided with holes 104 of different size and shape. Some holes are circular with different diameter where the holes 104 closest to the exposed part of the suction tube or line are portion are the smallest. Other holes are elongated.

The absorption body 111 is made of suitably elastically deformable material which enables fluid transport through the body, preferably foam plastic with open cells. It is also possible to use other soft materials that have capillary action like for example, cotton fibers, various textile fibers, synthetic fibers, mull, lint or gauze, preferably covered by a mesh, and the like.

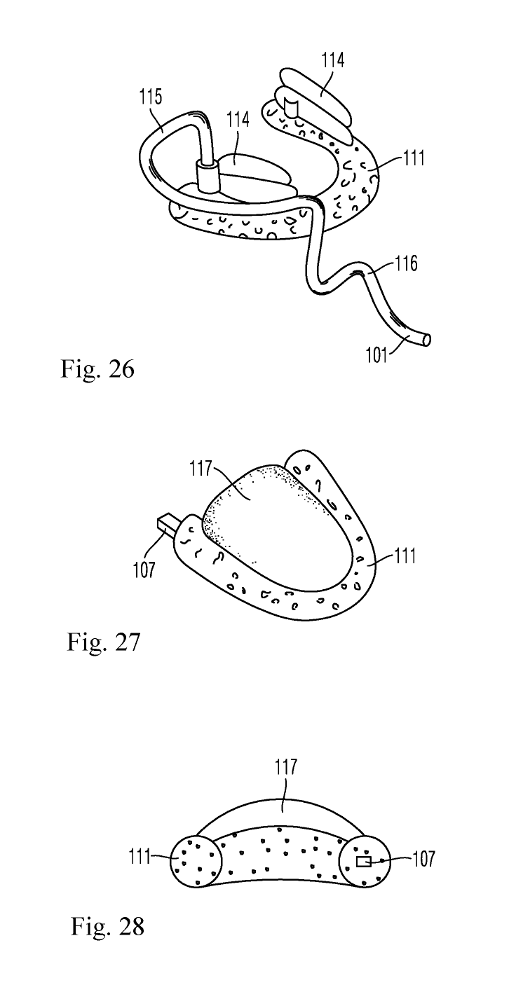

In the embodiment example illustrated in FIG. 26 the suction device comprises a tongue protection 114. The tongue protection aims to block the tongue and so protect the tongue from rotating instruments during the dental treatment and also to prevent the tongue from touching the tooth or teeth in any critical area which may cause a contamination with saliva when performing different filling and/or cementation of prosthetic constructions. FIG. 26 illustrates an embodiment, where two tongue protections 114 are arranged on both end sides of the absorption body 111. In this embodiment they are connected to the suction part 103 and extend through the foam of the absorption body 111, but in another embodiment, the tongue protection 114 may be arranged on one sidearm of an exposed suction tube 120. In the present embodiment, the suction device 101 includes two tongue protections, one arranged on one sidearm 120 an one arranged on the exposed suction tube 102. Two tongue protections can be made of elastically deformable material. Thus, the tongue protection can be bent, and therefore effectively separate the tongue from the operation, while also allowing the assisting personal to obtain full access and insight for required interventions and complementary removal of water and saliva.

In one embodiment, the tongue protection is made of a material which can be broken off, in order to adjust the fitting and placement in the oral cavity and also in order to enable an increased access and insight while the ability to protect the tongue during treatment is retained.

Furthermore, the tongue protection may be arranged in relation to the suction part 103 of the suction tube 101 and the absorption body 111.

In a further (not illustrated) embodiment, the tongue protection has an absorption capacity. This absorption capacity can either be provided in case the tongue protection is made of absorbable material or when the tongue protection is equipped with a suction tube which is connected with the suction device and its absorption body.

As may be taken from FIG. 26 (and FIG. 22), the suction tube 101 or suction line 101 extends upwardly from the absorption body 111. It is intended to transverse the lower jaw distal from the last molar, there crossing a tooth-free and gingival portion of the lower jaw. This part is designated as transverse part 115 of the suction tube 101. The suction line 101 then enters the user's mouth downwardly along the user's chin. The suction line comprises a counter bow 116 which is intended to be pressed against the user's chin from its lower side such as to keep the absorption body 111 in place.

Another embodiment is shown in FIG. 27. It has a plug 107 which is intended to be attached to a sleeve 106 of the exposed part 102 of a suction line 101.

The absorption body 111 is essentially parabolic or essentially U-shaped. Contrary to the remaining embodiments, this embodiment comprises a tongue shield 117 which is intended to keep the tongue at its regular position and to avoid tongue play which would unduly disturb surgery by the dentist.

The tongue shield 117 may be made of any suitable material and also may be very elastic. The tongue shield may be attached to the absorption body 111 but also to the suction part 103 running through the absorption body 111.

A back view, i. e. a distal view, of this embodiment is shown in FIG. 28.

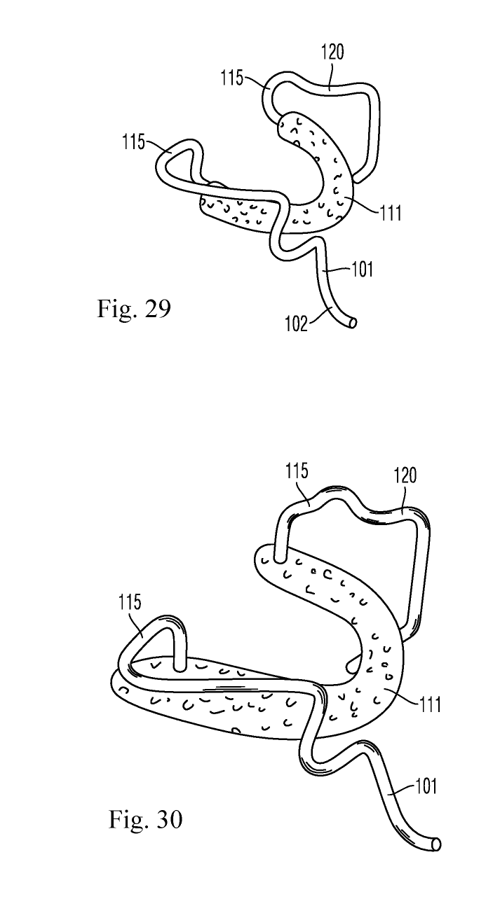

FIG. 29 shows another embodiment comprising a U-shaped or parabolic shaped absorption body 111 and a suction line 101, with its exposed part 102 being shown.

Contrary to other embodiments, there is an exposed suction tube 120 extending from the other hand of the absorption body 111. This exposed part is intended to also comprise a transverse part 115. The exposed part 120 is shaped in a suitable manner to keep the absorption body 111 in place by bi-lateral fixation under the chin. The suction line 101 exiting from the mouth may have any suitable form.

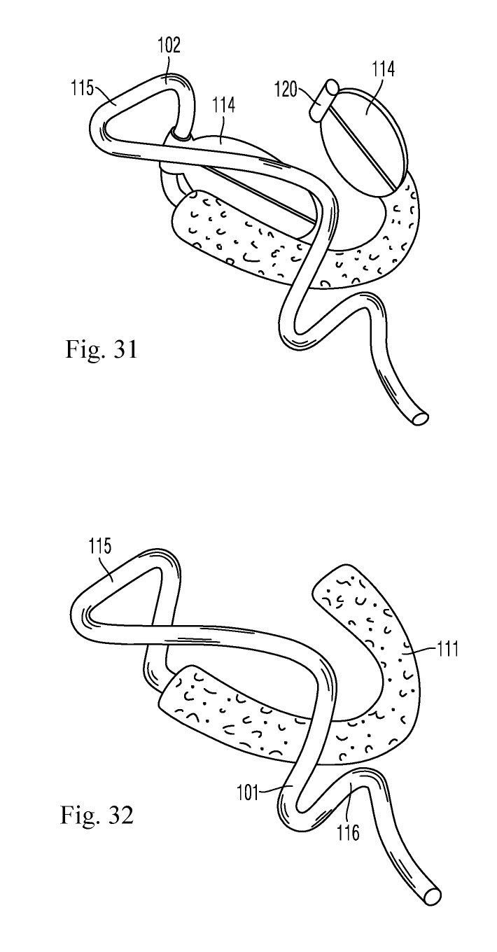

Another example is shown in FIG. 32. With this example, the counter bow 116 is intended to be arranged centrally under the user's chin, thus to make the downward pressing force of the suction line towards the absorption body 111 having a less unilateral attack angle.

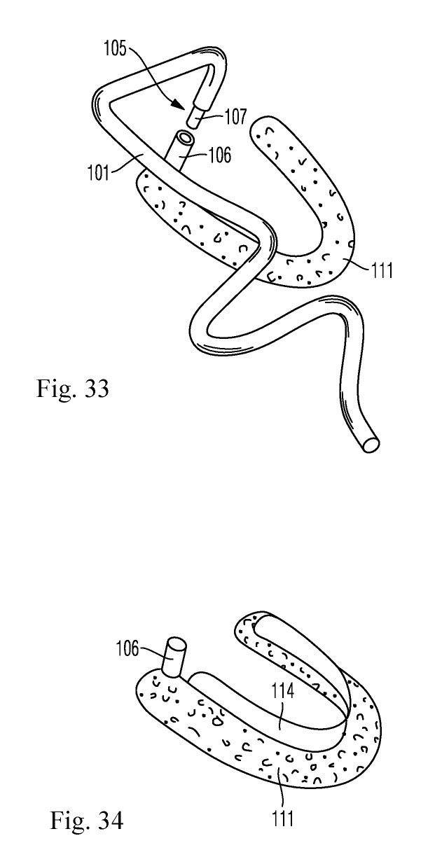

A similar arrangement may be taken from FIG. 33. In this arrangement, a junction 105 comprises with a sleeve 106 and a plug 107. Contrary to FIGS. 2a and 2b, the sleeve 106 with this embodiment is arranged close to the absorption body 111.

In FIG. 34, another embodiment of an absorption body 111 is shown. This embodiment comprises a tongue shield 114 which extends upwardly from the absorption body 111 and has a parabolic shape intended to keep away the tongue from the dental arch.

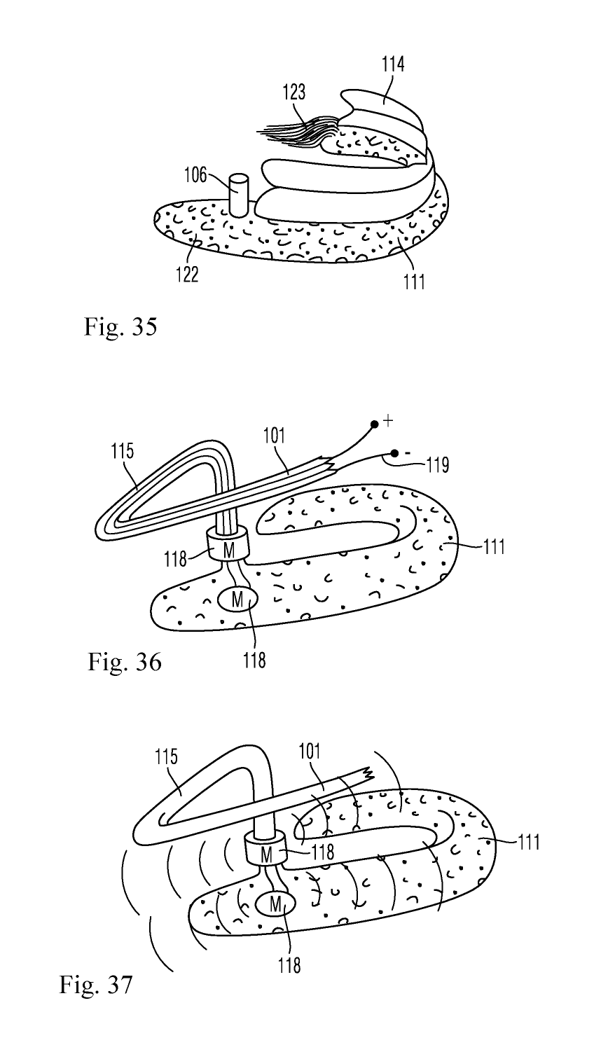

In FIG. 35, the same as 11A but with the extension parts shown 122 as a part of the foam body and 123 as a separate connected part of mesh or fibres or similar.

Advantageously, at least one protection shield is used which is attached to the absorption body 111, the suction part 103 and/or the exposed part 102 of the suction line 101, or a free end thereof. The protection shield 114 or the protection shields 114 may be bendable and can be brought into any desired form. They may have at least one breaking line, and/or may be light cure shields.

FIG. 36 shows another embodiment which comprises a moisture sensor 118 which is attached to above absorption body 111 or mounted within the absorption body 111 which is shown schematically in FIG. 12. There are supply lines 119 running through the suction line 101 for electrical connection of the moisture sensor 118.

FIG. 37 shows another embodiment which comprises a moisture sensor 118 which is attached to above absorption body 111 or mounted within the absorption body 111 which is shown schematically in FIG. 37. These sensors are transmitting wireless to a suitable device, e.g. the vacuum-system.

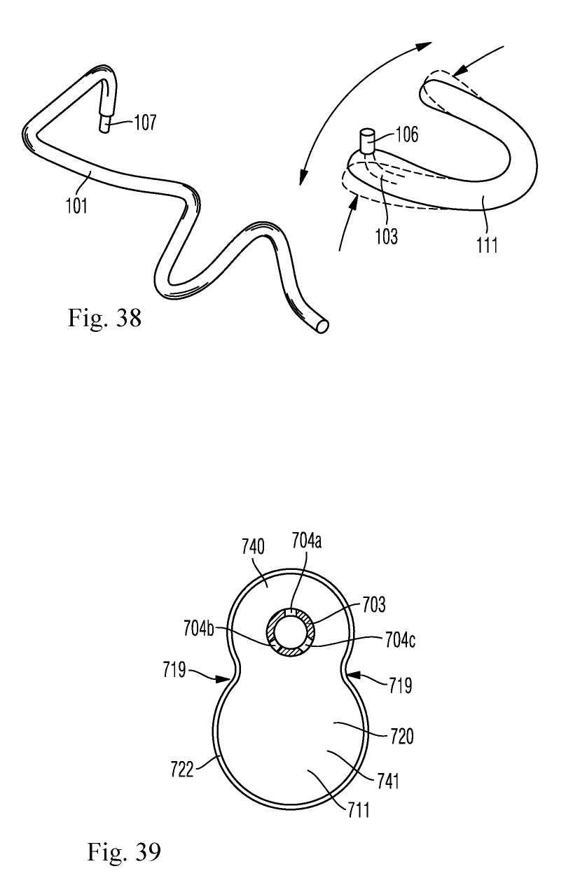

From FIG. 38, it may be taken that the absorption body 111 comprising the suction part 103 may be bent to any suitable form, within the anatomy as desired.

If needed it may be also be shorted by cutting off its ends or at least one end thereof.

The absorption body 111 is made by open-porous foam with a high elasticity, i.e. a soft open foam. This foam, according to the shown embodiment, is covered by a silky mesh which is also open-porous, to let the fluid pass, and is pleasant and comfortable to the patient. Alternatively, it is closed-porous to block fluids.

FIG. 39 shows another embodiment of the inventive dental suction arrangement.

A suction part 703 passes through the top portion 740. It has a plurality of suction holes 704a, 704b and 704c which are distributed uneven and with different diameters and form all over the suction part 703.

Preferably, the suction part 703 extends in the center of the top portion 740. In the different embodiment it may extend eccentrically, in order to increase the suction power at one side of the absorption body 711.

The idea is to have the absorption body 711 being arranged as low as possible under the patient's tongue in the bottom of the mouth, close to glandolar submandibularis and glandolar sublingualis, and a tongue holder extending upwardly from there and pressing the tongue obliquely upwardly and way from the jaw.

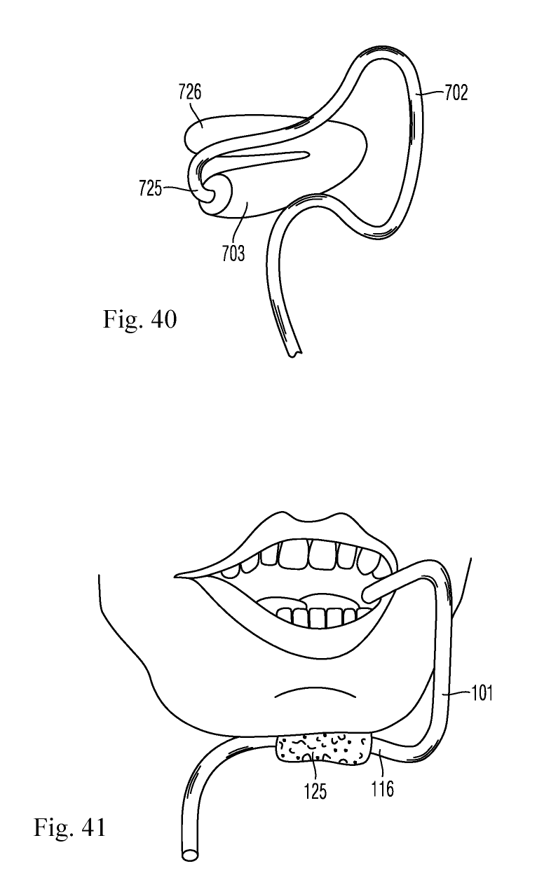

Another embodiment is shown in FIG. 40. This embodiment comprises a suction line 702 and a so-called “full jaw” absorption body 703. This absorption body is essentially U-shaped and comprises a suction part with suction holes even this is not shown in FIG. 40. It is intended to be placed under the tongue and the suction line 702 is of a flexibility such that it may be bent at a hinge area 725, to have this essentially U-shaped absorption body being inserted into the oral cavity either such that the suction line 702 is arranged on the left side or on the right side.

According to the invention, the absorption body 111 is placed under the tongue, following the essential parabolic shape of the dental arch at the oral side. The absorption foam body is of a open porous foam such that it may be compressed between the tongue and the dental arch to up to 85 volume percent. A transverse region 115 of the suction line 101 is provided which extends essentially horizontal for crossing the jaw after the last molar on one side of the mouth or on both sides of the mouth The transverse portion 115 is caught between upper and lower jaw, thus indirectly fixing the absorption body 111. The absorption body 111 is fixed especially in occlusion but is pressed downward even before occlusion by the flexible suction line 101.

For further improving the fixture, a counter bow 116 is provided in the suction line 101 which is intended to further fix it under the user's chin.

According to FIG. 41, this counter bow 116 may be provided with an additional foam body 115 for improving user's comfort.

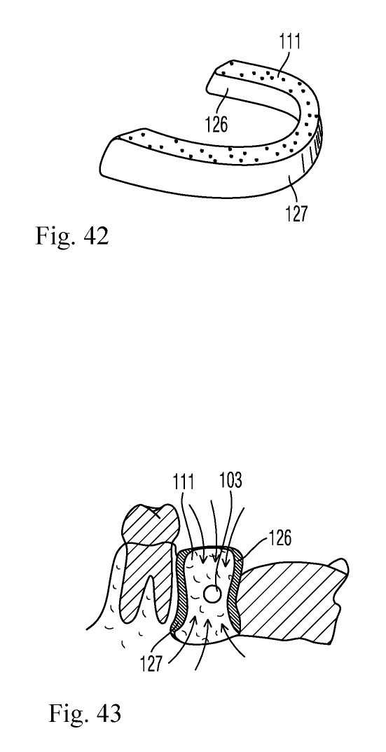

A further embodiment of the absorption body 111 is shown in FIGS. 42 and 43. In this embodiment, the absorption body is provided with a silky mesh 126 and 127 on both the vestibular side and the oral side.

Thus, as may be taken from FIG. 43, the suction force is focused both on the upper side and the lower side of the absorption body 111 which is beneficial to improve suction efficiency.

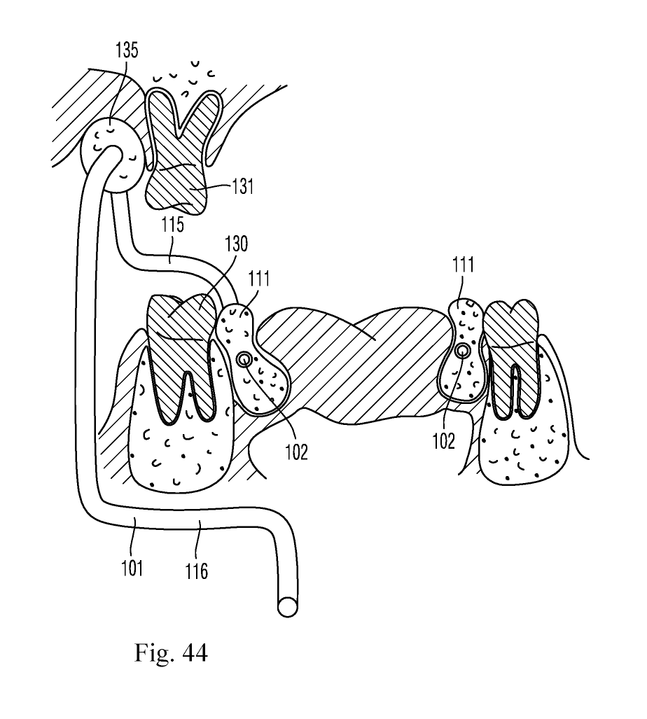

Another embodiment of the invention is shown in FIG. 44. This embodiment is shown, for the purpose of clarity, before occlusion, i.e. with antagonists still being separate from another. Yet it is intended to be functional in occlusion, with the transverse portion 115 extending distal from or behind the last molar 130.

The suction line 101, when exiting the transverse portion 115, is bent upwardly and is provided with an additional foam body 135 which is intended to be arranged vestibular of the antagonist 131. From this area, the suction line 101 runs downwardly and form a counter bow 116 as shown in other embodiments.

The invention has been described in relation to the current understanding of which are the most practical and preferred embodiments, but it is recognized that the invention is not limited to the described embodiments; several variations and modifications are possible. The scope of the invention is therefore exclusively defined by the attached patent claims.

1. Dental suction arrangement for the removal of saliva and other fluids—comprising
a suction line (101, 602, 702) and
an absorption body (111, 611, 711) to be placed for the removal of saliva and other fluids,
whereby the suction line (101) comprises a suction part (103) which is arranged in the absorption body (111),
wherein the suction part (103) is equipped with hole members (104, 904a-e) communicating with the absorption body,
wherein the suction line (101, 602, 702) has a prefabricated form and size,
wherein the suction line (101,602,702) extends a distance from the absorption body for secure placement,
wherein said hole members (104, 904a-e) are distributed along a longitudinal extent of said suction part (103) and along a periphery of said suction part, and
wherein the absorption body (111, 611, 711) is made of elastically deformable and adaptable material in order to adapt the absorption body to a desired shape.

2. Dental suction arrangement according to claim 1,
wherein the absorption body (111, 611, 711), in a cross sectional view perpendicular to the longitudinal extent of the absorption body, comprises a contour of at least one of the following: a non-circular contour, a contour with a base and a top, whereby the base is broader than the top, and/or a contour with at least one concave part (119).

3. Dental suction arrangement according to claim 1,
wherein the absorption body (111, 611, 711) includes an extension part (113, 613) which, when placed in the cavity, extends distal from the suction line (101, 601,701), whereby the extension part (113, 613) has a reduced cross-sectional area which decreases in the direction of its distal end.

4. Dental suction arrangement according to claim 1,
wherein the absorption body (111, 611, 711) is adjustable in length by being ductily deformed, and/or has a base body with a punch pattern allowing said body together with a foam body of the absorption body to be extended.

5. Dental suction arrangement according to claim 1,
wherein the absorption body (111, 611, 711) is surrounded by or made of a silky mesh, or a soft flocking, said mesh or flocking having a capillary effect.

6. Dental suction arrangement according to claim 1,
wherein said hole members comprise holes (104, 904a-e) of different sizes and/or different forms.

7. Dental suction arrangement according to claim 1,
further comprising a secondary and adjustable suction line (605) extending from the main absorption body (602, 603, 611) for connection with a secondary absorption body (618).

8. Dental suction arrangement according to claim 1,
wherein the suction part (103) is detachably connected with an exposed portion of the suction line (102) together with an absorption body (111) associated with the suction part.

9. Dental suction arrangement according to claim 1,
wherein the absorption body (111, 611, 711) and the suction part (103) which is disposed inside the absorption body, are essentially U-shaped,
said suction line (101, 601,701) exiting from the absorption body (111, 611, 711) at an end thereof.

10. Dental suction arrangement according to claim 1,
further comprising at least one protection shield (714) for withholding a third part from moving toward the dental suction arrangement.

11. Dental suction arrangement according to claim 10,
wherein the at least one protection shield (714) is arranged at an extension of the exposed portion of the suction line (102) and/or
wherein the at least one protection shield (714) is arranged on a sidearm of the exposed portion of the suction line (102), and/or
wherein the inside suction part (103) and protection shield (714) are detachably connected with the exposed portion of the suction line (102).

12. Dental suction arrangement according to claim 1,
further comprising an exposed portion of the suction line (102) and a set of multiple absorption bodies (111, 611, 711) in different shapes and/or sizes, and a set of multiple protection shields (714) in different shapes and/or sizes.

13. Dental suction arrangement according to claim 10,
wherein the at least one protection shield (714) is adjustable and especially bendable and/or has at least one hinge and/or has at least one predetermined breaking point.

14. Dental suction arrangement according to claim 10,
wherein the at least one protection shield (714) is mounted on a second suction line (705) distal from said absorption body (711), said second suction line extending along a length of said absorption body (711), said second suction line (705) being adjustable for any alignment of the protection shield (714) at a user's discretion.

15. Dental suction arrangement according to claim 10,
wherein the protection shield (714) forms a light curing shield and is made from orange acrylic material.

16. Dental suction arrangement according to claim 10,
wherein the protection shield (714) has a flat and elongated shape, and extends as an extension of said suction line (705) and/or has a protrusion extending laterally from the elongated shape.

17. Dental suction arrangement according to claim 18,
wherein said cavity is an oral cavity,
wherein said elevated portion is a lower jaw,
wherein said protection shield is a tongue protection,
wherein a secondary absorption body (618) is arranged for being positioned at the parotis gland duct, and/or
wherein the bottom is the area between a tongue and the bottom of a mouth.

18. Dental suction arrangement according to claim 1,
wherein the suction line (101, 602, 702) has a prefabricated form and size for positioning said absorption body (111) at the bottom of a cavity, or close to said bottom, and said suction line (101,602,702) extends along an elevated portion upward from said bottom, and at least partially surrounding said portion for secure fixing.

19. Dental suction arrangement according to claim 7,
wherein the secondary and adjustable suction line (605) comprises multiple absorption bodies and additional suction lines, whereby each additional suction line is connected with a suction part in a respective absorption body.

20. Dental suction arrangement according to claim 8, further comprising
a set of detachable suction lines having absorption bodies of varying shape and/or size.
